(19) United States
(12) Patent Application Publication
(10) Pub. No.: US 2026/0248573 A1
(43) Pub. Date: Aug. 27, 2026

(54) CHANGING MODE OF OPERATION OF AN INSTRUMENT BASED ON GESTURE DETECTION

(71) Applicant: INTUITIVE SURGICAL OPERATIONS, INC., Sunnyvale, CA (US)

(72) Inventors: Yidan QIN, Pasadena, CA (US); Maximilian Hunter ALLAN, Mill Valley, CA (US); Craig R. GERBI, Half Moon Bay, CA (US); Scott E. MANZO, Shelton, CT (US); Liliann MUELLER, New Haven, CT (US); Andrej SIMEUNOVIC, Stanford, CA (US)

(21) Appl. No.: 18/995,432

(22) PCT Filed: Jul. 21, 2023
(86) PCT No.: PCT/US2023/028412

§ 371 Date: Jan. 16, 2025

Related U.S. Application Data
(60) Provisional application No. 63/391,418, filed on Jul. 22, 2022.

(51) Int. Cl. A61B 34/37; A61B 17/00; A61B 90/00

(52) U.S. Cl. CPC A61B 34/37; A61B 17/00; A61B 90/361; A61B 2017/00207

(57) ABSTRACT

Systems and methods for changing a mode of operation of an instrument includes a computer-assisted device configured to obtain vision data associated with an instrument; obtain kinematic data associated with at least one of the instrument or an structure supporting the instrument; obtain event data associated with at least one of the structure or the instrument; based on the vision data, the kinematic data, and the event data, recognize a gesture performed via the instrument; and in response to recognizing the gesture, cause the computer-assisted device to change from a first mode of operation to a second mode of operation.

### RELATED APPLICATIONS

This application claims the benefit to U.S. Provisional Application No. 63/391,418, filed Jul. 22, 2022, and entitled “Changing Mode of Operation of an Instrument Based On Gesture Detection,” the subject matter of which is incorporated by reference herein.

### BACKGROUND

### Field of the Various Embodiments

The present disclosure is directed to operation of instruments associated with computer-assisted devices, and more particularly to techniques for changing a mode of operation of an instrument associated with a computer-assisted device based on detection of gestures.

### Description of the Related Art

More and more devices are being replaced with computer-assisted electronic devices. This is especially true in industrial, entertainment, educational, and other settings. As a medical example, the hospitals of today include large arrays of electronic devices being found in operating rooms, interventional suites, intensive care wards, emergency rooms, and/or the like. For example, glass and mercury thermometers are being replaced with electronic thermometers, intravenous drip lines now include electronic monitors and flow regulators, and traditional hand-held surgical and other medical instruments are being replaced by computer-assisted medical devices.

These computer-assisted devices are useful for performing operations and/or procedures on materials, such as the tissue of a patient. With many computer-assisted devices, an operator, such as a surgeon and/or other medical personnel, may typically manipulate input devices using one or more controls on an operator console. As the operator operates the various controls at the operator console, the commands are relayed from the operator console to a computer-assisted device located in a workspace where they are used to position and/or actuate one or more end effectors and/or tools that are supported (e.g., via repositionable arms) by the computer-assisted device. In this way, the operator is able to perform one or more procedures on material in the workspace using the end effectors and/or tools.

Each of the one or more end effectors and/or tools can perform multiple functions, have multiple modes of operation, and/or operate according to one or more adjustable parameters. In a medical example, a tissue sealing instrument can operate in different energy modes depending on the needs of the operator and the task at hand. During a procedure, the operator of the computer-assisted device can change the operating functionality, mode, and/or parameter of the instrument.

Current approaches to facilitating changes to a functionality, mode, and/or parameter of an instrument associated with a computer-assisted device by the operator include using additional physical inputs and/or a graphical user interface. For example, the computer-assisted device can include foot pedals that can be assigned a capability to change the mode of operation of an instrument. As another example, the operator can select a mode from a graphical user interface on a display. However, these approaches make adding instruments and modes to the computer-assisted device difficult. If an instrument with multiple modes is to be added to the computer-assisted device, in order to facilitate mode changes for that instrument, the computer-assisted device could be modified to include additional physical inputs (e.g., additional buttons, foot pedals, and/or the like; adding voice input capability were none existed before) and/or additional options in the graphical user interface. Any of these options would require modification of the hardware and/or software of the computer-assisted device. These additional inputs and/or options would add to the learning curve of the operator, who would need to need to adjust to the new inputs and/or options.

Further, the current approaches to changing mode, etc. can be disruptive to the workflow of the operator. In particular, the current approaches require the operator to operate an input device or user interface that are not part of the procedure workflow but for the capability of the input device or user interface to change the operation of the instrument. The attention of the operator is distracted from the workflow toward the input device or user interface, reducing the situational awareness of the operator with respect to the procedure workflow.

Accordingly, improved methods and systems for modifying the operation of an instrument associated with a computer-assisted device are desirable. In some examples, it may be desirable to provide gesture-based changes in the operating functionality, mode, and/or parameter of the instrument, so as to help ensure that the instrument may be able to successfully perform a desired procedure.

### SUMMARY

Consistent with some embodiments, a computer-assisted device comprises a structure configured to support an instrument, memory storing an application, and a processing system. When executing the application, the processing system is configured to obtain kinematics data associated with at least one of the structure or the instrument based on at least the kinematics data, recognize a gesture performed via the instrument; and in response to recognizing the gesture, cause the computer-assisted device to change from a first mode of operation to a second mode of operation.

Consistent with some embodiments, a method comprises obtaining kinematics data associated with at least one of an instrument or a structure supporting the instrument; based on at least the kinematics data, recognizing a gesture performed via the instrument; and in response to recognizing the gesture, causing a change from a first mode of operation to a second mode of operation.

Consistent with some embodiments, a computer-assisted device comprises a structure configured to support an instrument, memory storing an application, and a processing system. When executing the application, the processing system is configured to obtain vision data associated with the instrument; obtain kinematics data associated with at least one of the structure or the instrument; obtain events data associated with at least one of the structure or the instrument; based on the vision data, the kinematics data, and the events data, recognize a gesture performed via the instrument; and in response to recognizing the gesture, cause the computer-assisted device to change from a first mode of operation to a second mode of operation.

Consistent with some embodiments, a method comprises obtaining vision data associated with an instrument; obtaining kinematics data associated with at least one of the instrument or a structure supporting the instrument; obtaining events data associated with at least one of the structure or the instrument; based on the vision data, the kinematics data, and the events data, recognizing a gesture performed via the instrument; and in response to recognizing the gesture, causing a change from a first mode of operation to a second mode of operation.

Consistent with some embodiments, one or more non-transitory machine-readable media include a plurality of machine-readable instructions which when executed by a processor system associated with a computer-assisted system are adapted to cause the processor system to perform any of the methods described herein.

At least one advantage and technical improvement of the disclosed techniques relative to the prior art is that, with the disclosed techniques, the mode or functionality of an instrument can be changed without significant diversion from an on-going procedure. Accordingly, the operator can maintain a high situational awareness with respect to the on-going procedure. Another advantage and technical improvement is that new instruments with multiple modes and/or functions can be added to a computer-assisted device without significant operator-facing modifications to the computer-assisted device or the user interface. Accordingly, a computer-assisted device can be expanded to include new instruments transparently and without a significant learning curve for the operator. These technical advantages provide one or more technological advancements over prior art approaches.

### BRIEF DESCRIPTION OF THE DRAWINGS

So that the manner in which the above recited features of the various embodiments can be understood in detail, a more particular description of the inventive concepts, briefly summarized above, may be had by reference to various embodiments, some of which are illustrated in the appended drawings. It is to be noted, however, that the appended drawings illustrate only typical embodiments of the inventive concepts and are therefore not to be considered limiting of scope in any way, and that there are other equally effective embodiments.

FIG. 1 is a is a simplified diagram including an example of a computer-assisted system, according to various embodiments.

FIG. 2 illustrates the control module of FIG. 1 in greater detail, according to various embodiments.

FIG. 3 illustrates the mode change module of FIG. 2 in greater detail, according to various embodiments.

FIGS. 4A-4C illustrates an example machine learning implementation of the mode change module of FIG. 3 according to some embodiments.

FIG. 5 is a table illustrating example active and passive gestures according to some embodiments.

FIG. 6 is a flow chart of method steps for modifying the operation of an instrument, according to some embodiments.

### DETAILED DESCRIPTION

This description and the accompanying drawings that illustrate inventive aspects, embodiments, embodiments, or modules should not be taken as limiting—the claims define the protected invention. Various mechanical, compositional, structural, electrical, and operational changes may be made without departing from the spirit and scope of this description and the claims. In some instances, well-known circuits, structures, or techniques have not been shown or described in detail in order not to obscure the invention. Like numbers in two or more figures represent the same or similar elements.

In this description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional.

Further, the terminology in this description is not intended to limit the invention. For example, spatially relative terms—such as “beneath”, “below”, “lower”, “above”, “upper”, “proximal”, “distal”, and the like—may be used to describe one element's or feature's relationship to another element or feature as illustrated in the figures. These spatially relative terms are intended to encompass different positions (i.e., locations) and orientations (i.e., rotational placements) of the elements or their operation in addition to the position and orientation shown in the figures. For example, if the content of one of the figures is turned over, elements described as “below” or “beneath” other elements or features would then be “above” or “over” the other elements or features. Thus, the exemplary term “below” can encompass both positions and orientations of above and below. A device may be otherwise oriented (rotated 90 degrees or at other orientations) and the spatially relative descriptors used herein interpreted accordingly. Likewise, descriptions of movement along and around various axes include various special element positions and orientations. In addition, the singular forms “a”, “an”, and “the” are intended to include the plural forms as well, unless the context indicates otherwise. And, the terms “comprises”, “comprising”, “includes”, and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components.

Elements described in detail with reference to one embodiment, implementation, or module may, whenever practical, be included in other embodiments, embodiments, or modules in which they are not specifically shown or described. For example, if an element is described in detail with reference to one embodiment and is not described with reference to a second embodiment, the element may nevertheless be claimed as included in the second embodiment. Thus, to avoid unnecessary repetition in the following description, one or more elements shown and described in association with one embodiment, embodiment, or application may be incorporated into other embodiments, embodiments, or aspects unless specifically described otherwise, unless the one or more elements would make an embodiment or embodiment non-functional, or unless two or more of the elements provide conflicting functions.

In some instances, well known methods, procedures, components, and circuits have not been described in detail so as not to unnecessarily obscure aspects of the embodiments.

This disclosure describes various elements (such as systems and devices, and portions of systems and devices) in three-dimensional space. As used herein, the term “position” refers to the location of an element or a portion of an element in a three-dimensional space (e.g., three degrees of translational freedom along Cartesian x-, y-, and z-coordinates). As used herein, the term “orientation” refers to the rotational placement of an element or a portion of an element (three degrees of rotational freedom—e.g., roll, pitch, and yaw). As used herein, the term “pose” refers to the multi-degree of freedom (DOF) spatial position and/or orientation of a coordinate system of interest attached to a rigid body. In general, a pose can include a pose variable for each of the DOFs in the pose. For example, a full 6-DOF pose would include 6 pose variables corresponding to the 3 positional DOFs (e.g., x, y, and z) and the 3 orientational DOFs (e.g., roll, pitch, and yaw). A 3-DOF position only pose would include only pose variables for the 3 positional DOFs. Similarly, a 3-DOF orientation only pose would include only pose variables for the 3 rotational DOFs. Poses with any other number of DOFs (e.g., one, two, four, or five) are also possible. As used herein, the term “shape” refers to a set positions or orientations measured along an element. As used herein, and for an element or portion of an element, e.g., a device (e.g., a computer-assisted system or a repositionable arm), the term “proximal” refers to a direction toward the base of the system or device of the repositionable arm along its kinematic chain, and the term “distal” refers to a direction away from the base along the kinematic chain.

Aspects of this disclosure are described in reference to computer-assisted systems, which may include systems and devices that are teleoperated, remote-controlled, autonomous, semiautonomous, manually manipulated, and/or the like. Example computer-assisted systems include those that comprise robots or robotic devices. Further, aspects of this disclosure are described in terms of an embodiment using a medical system, such as the da Vinci® Surgical System commercialized by Intuitive Surgical, Inc. of Sunnyvale, California. Knowledgeable persons will understand, however, that inventive aspects disclosed herein may be embodied and implemented in various ways, including robotic and, if applicable, non-robotic embodiments. Embodiments described for da Vinci® Surgical Systems are merely exemplary, and are not to be considered as limiting the scope of the inventive aspects disclosed herein. For example, techniques described with reference to surgical instruments and surgical methods may be used in other contexts. Thus, the instruments, systems, and methods described herein may be used for humans, animals, portions of human or animal anatomy, industrial systems, general robotic, or teleoperational systems. As further examples, the instruments, systems, and methods described herein may be used for non-medical purposes including industrial uses, general robotic uses, sensing or manipulating non-tissue work pieces, cosmetic improvements, imaging of human or animal anatomy, gathering data from human or animal anatomy, setting up or taking down systems, training medical or non-medical personnel, and/or the like. Additional example applications include use for procedures on tissue removed from human or animal anatomies (with or without return to a human or animal anatomy) and for procedures on human or animal cadavers. Further, these techniques can also be used for medical treatment or diagnosis procedures that include, or do not include, surgical aspects.

### System Overview

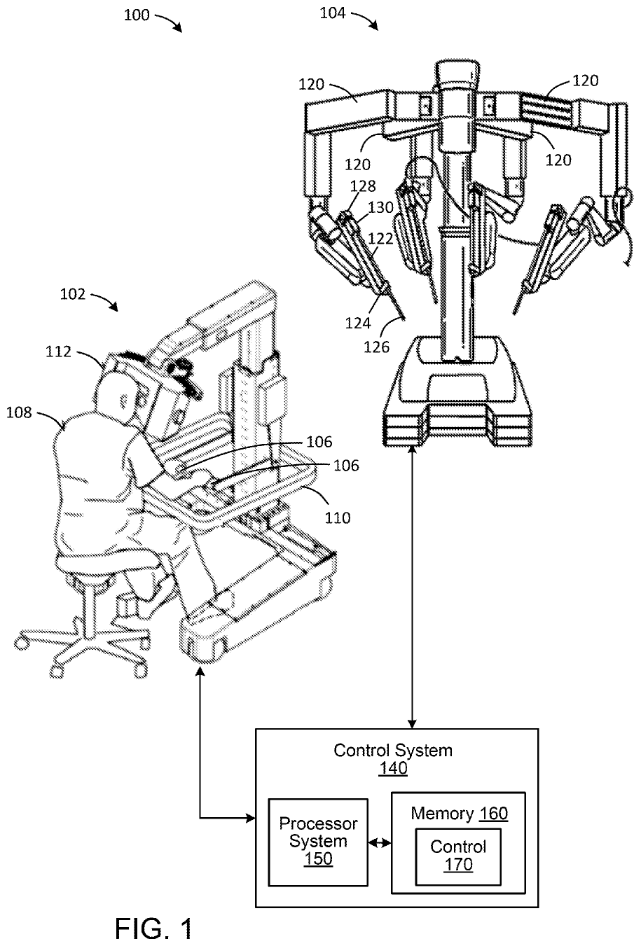

FIG. 1 is a simplified diagram of an example computer-assisted system 100, according to various embodiments. In some examples, the computer-assisted system 100 is a teleoperated system. In medical examples, computer-assisted system 100 can be a teleoperated medical system such as a surgical system. As shown, computer-assisted system 100 includes a follower device 104 that can be teleoperated by being controlled by one or more leader devices (also called “leader input devices” when designed to accept external input), described in greater detail below. Systems that include a leader device and a follower device are referred to as leader-follower systems, and also sometimes referred to as master-slave systems. Also shown in FIG. 1 is an input system that includes a workstation 102 (e.g., a console), and in various embodiments the input system can be in any appropriate form and may or may not include a workstation 102.

In the example of FIG. 1, workstation 102 includes one or more leader input devices 106 that are designed to be contacted and manipulated by an operator 108. For example, workstation 102 can comprise one or more leader input devices 106 for use by the hands, the head, or some other body part(s) of operator 108. Leader input devices 106 in this example are supported by workstation 102 and can be mechanically grounded. In some embodiments, an ergonomic support 110 (e.g., forearm rest) can be provided on which operator 108 can rest his or her forearms. In some examples, operator 108 can perform tasks at a worksite near follower device 104 during a procedure by commanding follower device 104 using leader input devices 106.

A display unit 112 is also included in workstation 102. Display unit 112 can display images for viewing by operator 108. Display unit 112 can be moved in various degrees of freedom to accommodate the viewing position of operator 108 and/or to optionally provide control functions as another leader input device. In the example of computer-assisted system 100, displayed images can depict a worksite at which operator 108 is performing various tasks by manipulating leader input devices 106 and/or display unit 112. In some examples, images displayed by display unit 112 can be received by workstation 102 from one or more imaging devices arranged at a worksite. In other examples, the images displayed by display unit 112 can be generated by display unit 112 (or by a different connected device or system), such as for virtual representations of tools, the worksite, or for user interface components.

When using workstation 102, operator 108 can sit in a chair or other support in front of workstation 102, position his or her eyes in front of display unit 112, manipulate leader input devices 106, and rest his or her forearms on ergonomic support 110 as desired. In some embodiments, operator 108 can stand at the workstation or assume other poses, and display unit 112 and leader input devices 106 can be adjusted in position (height, depth, etc.) to accommodate operator 108.

In some embodiments, the one or more leader input devices 106 can be ungrounded (ungrounded leader input devices being not kinematically grounded, such as leader input devices held by the hands of operator 108 without additional physical support). Such ungrounded leader input devices can be used in conjunction with display unit 112. In some embodiments, operator 108 can use a display unit 112 positioned near the worksite, such that operator 108 manually operates instruments at the worksite, such as a laparoscopic instrument in a surgical example, while viewing images displayed by display unit 112.

Computer-assisted system 100 also includes follower device 104, which can be commanded by workstation 102. In a medical example, follower device 104 can be located near an operating table (e.g., a table, bed, or other support) on which a patient can be positioned. In some medical examples, the worksite is provided on an operating table, e.g., on or in a patient, simulated patient, or model, etc. (not shown). The follower device 104 shown includes a plurality of manipulator arms 120, each manipulator arm 120 configured to couple to an instrument assembly 122. An instrument assembly 122 can include, for example, an instrument 126. In various embodiments, examples of instruments 126 include, without limitation, a sealing instrument, a cutting instrument, a sealing-and-cutting instrument, a radio frequency energy delivery instrument, an ultrasonic energy delivery instrument, a suturing instrument (e.g., a suturing needle), a needle instrument (e.g., a biopsy needle), or a gripping or grasping instrument (e.g., clamps, jaws), a suction and/or irrigation instrument, and/or the like. As shown, each instrument assembly 122 is mounted to a distal portion of a respective manipulator arm 120. The distal portion of each manipulator arm 120 further includes a cannula mount 124 which is configured to have a cannula (not shown) mounted thereto. When a cannula is mounted to the cannula mount, a shaft of an instrument 126 passes through the cannula and into a worksite, such as a surgery site during a surgical procedure. A force transmission mechanism 130 of the instrument assembly 122 can be connected to an actuation interface assembly 128 of the manipulator arm 120 that includes drive and/or other mechanisms controllable from workstation 102 to transmit forces to the force transmission mechanism 130 to actuate the instrument 126.

In various embodiments, one or more of instruments 126 can include an imaging device for capturing images (e.g., optical cameras, hyperspectral cameras, ultrasonic sensors, endoscopes, etc.). For example, one or more of instruments 126 can be an endoscope assembly that includes an imaging device, which can provide captured images of a portion of the worksite to be displayed via display unit 112.

In some embodiments, the manipulator arms 120 and/or instrument assemblies 122 can be controlled to move and articulate instruments 126 in response to manipulation of leader input devices 106 by operator 108, and in this way “follow” the leader input devices 106 through teleoperation. This enables the operator 108 to perform tasks at the worksite using the manipulator arms 120 and/or instrument assemblies 122. Manipulator arms 120 are examples of repositionable structures that a computer-assisted device (e.g., follower device 104) can include. In some embodiments, a repositionable structure of a computer-assisted device can include a plurality of links that are rigid members and joints that are movable components that can be actuated to cause relative motion between adjacent links. For a surgical example, the operator 108 can direct follower manipulator arms 120 to move instruments 126 to perform surgical procedures at internal surgical sites through minimally invasive apertures or natural orifices.

As shown, a control system 140 is provided external to workstation 102 and communicates with workstation 102. In other embodiments, control system 140 can be provided in workstation 102 or in follower device 104. As operator 108 moves leader input device(s) 106, sensed spatial information including sensed position and/or orientation information is provided to control system 140 based on the movement of leader input devices 106. Control system 140 can determine or provide control signals to follower device 104 to control the movement of manipulator arms 120, instrument assemblies 122, and/or instruments 126 based on the received information and operator input. In one embodiment, control system 140 supports one or more wired communication protocols, (e.g., Ethernet, USB, and/or the like) and/or one or more wireless communication protocols (e.g., Bluetooth, IrDA, HomeRF, IEEE 1102.11, DECT, Wireless Telemetry, and/or the like).

Control system 140 can be implemented on one or more computing systems. One or more computing systems can be used to control follower device 104. In addition, one or more computing systems can be used to control components of workstation 102, such as movement of a display unit 112.

As shown, control system 140 includes a processor system 150 and a memory 160 storing a control module 170. In some embodiments, processor system 150 can include one or more processors, non-persistent storage (e.g., volatile memory, such as random access memory (RAM), cache memory), persistent storage (e.g., a hard disk, an optical drive such as a compact disk (CD) drive or digital versatile disk (DVD) drive, a flash memory, a floppy disk, a flexible disk, a magnetic tape, any other magnetic medium, any other optical medium, programmable read-only memory (PROM), an erasable programmable read-only memory (EPROM), a FLASH-EPROM, any other memory chip or cartridge, punch cards, paper tape, any other physical medium with patterns of holes, etc.), a communication interface (e.g., Bluetooth interface, infrared interface, network interface, optical interface, etc.), and numerous other elements and functionalities. The non-persistent storage and persistent storage are examples of non-transitory, tangible machine readable media that can include executable code that, when run by one or more processors (e.g., processor system 150), can cause the one or more processors to perform one or more of the techniques disclosed herein, including the process of method 600 described below. In addition, functionality of control module 170 can be implemented in any technically feasible software and/or hardware in some embodiments.

Each of the one or more processors of processor system 150 can be an integrated circuit for processing instructions. For example, the one or more processors can be one or more cores or micro-cores of a processor, a central processing unit (CPU), a microprocessor, a field-programmable gate array (FPGA), an application-specific integrated circuit (ASIC), a digital signal processor (DSP), a graphics processing unit (GPU), a tensor processing unit (TPU), and/or the like. Control system 140 can also include one or more input devices, such as a touchscreen, keyboard, mouse, microphone, touchpad, trackpad, electronic pen, or any other type of input device. In some embodiments, the one or more input devices are also used to help control instruments 126.

A communication interface of control system 140 can include an integrated circuit for connecting the computing system to a network (not shown) (e.g., a local area network (LAN), a wide area network (WAN) such as the Internet, mobile network, or any other type of network) and/or to another device, such as another computing system.

Further, control system 140 can include one or more output devices, such as a display device (e.g., a liquid crystal display (LCD), a plasma display, touchscreen, organic LED display (OLED), projector, or other display device), a printer, a speaker, external storage, or any other output device. One or more of the output devices can be the same or different from the input device(s). Many different types of computing systems exist, and the aforementioned input and output device(s) can take other forms.

In some embodiments, control system 140 can be connected to or be a part of a network. The network can include multiple nodes. Control system 140 can be implemented on one node or on a group of nodes. By way of example, control system 140 can be implemented on a node of a distributed system that is connected to other nodes. By way of another example, control system 140 can be implemented on a distributed computing system having multiple nodes, where different functions and/or components of control system 140 can be located on a different node within the distributed computing system. Further, one or more elements of the aforementioned control system 140 can be located at a remote location and connected to the other elements over a network.

Some embodiments can include one or more components of a teleoperated medical system such as a da Vinci® Surgical System, commercialized by Intuitive Surgical, Inc. of Sunnyvale, California, U.S.A. Embodiments on da Vinci® Surgical Systems are merely examples and are not to be considered as limiting the scope of the features disclosed herein. For example, different types of teleoperated systems having follower devices at worksites, as well as non-teleoperated systems, can make use of features described herein.

In some embodiments, control system 142 can record (e.g., log) system states and/or events taking place in computer-assisted system 100. A system state, as used herein, refers to any of: a state of computer-assisted system 100 and/or any component thereof (e.g., instrument 126, manipulator arms 120, an imaging device), any changes to the state of computer-assisted system 100 and/or any component thereof, identification and a current mode/functionality of an instrument 126 in current use, and/or a current parameter under which computer-assisted system 100 and/or a component thereof is operating (e.g., a level of grip force, a level of energy for sealing). An event, as used herein, refers to any of: any interaction between computer-assisted system 100 and a worksite (e.g., an action by instrument 126 on a target in the worksite, whether instrument 126 is contacting an object in the worksite), any action taken by an operator (e.g., operator 108) on computer-assisted system 100 and/or any component thereof (e.g., inputs made by operator 108 into computer-assisted system 100), any output made by computer-assisted system 100 and/or any component thereof (e.g., transmissions between workstation 102, control system 140, and follower device 104). For purposes of simplicity and brevity of this present disclosure, both system states and events are collectively referred to as events. In some embodiments, control module 170 generates an events log, records events in the events log, and stores the events log in a computer readable storage medium (e.g., memory 160).

Instrument 126 includes a proximal end and a distal end. In some embodiments, instrument 126 can have a flexible body. In some embodiments, instrument 126 includes, for example, an imaging device (e.g., an image capture probes), biopsy instrument, laser ablation fibers, and/or other medical surgical, diagnostic, or therapeutic tools. More generally, an instrument 126 can include an end effector and/or tool for performing a task. In some embodiments, a tool included in instrument 126 includes an end effector having a single working member, such as a scalpel, a blunt blade, an optical fiber, an electrode, and/or the like. Other end effectors may include, for example, forceps, graspers, scissors, clip appliers, and/or the like. Other end effectors may further include electrically activated end effectors such as electro surgical electrodes, transducers, sensors, and/or the like.

In some embodiments, instrument 126 can include a sealing instrument for sealing tissue (e.g., a vessel). A sealing instrument can operate according to any technically feasible sealing approach or technique, including for example bipolar sealing, monopolar sealing, or sealing and cutting sequentially or concurrently. Further, the sealing instrument can operate at any technically feasible energy level needed to perform the sealing operation. In some embodiments, an energy level parameter for instrument 126 can be configured or otherwise set by operator 108.

In some embodiments, instrument 126 can include a cutting instrument for cutting tissue. More generally, instrument 126 can include an instrument that can be operated by operator 108 to perform any suitable action in a procedure. In a medical context, such actions include but are not limited to sealing, cutting, gripping, stapling, applying a clip, irrigating, suturing, and so forth.

In some embodiments, instrument 126 can include an instrument that can perform different actions according to different modes, and/or perform an action according to different approaches and/or parameters. For example, a sealing instrument can operate according to a bipolar mode for bipolar sealing or a monopolar mode for monopolar sealing. As another example, a sealing and cutting instrument can include a first mode for sealing and cutting and a second mode for just sealing.

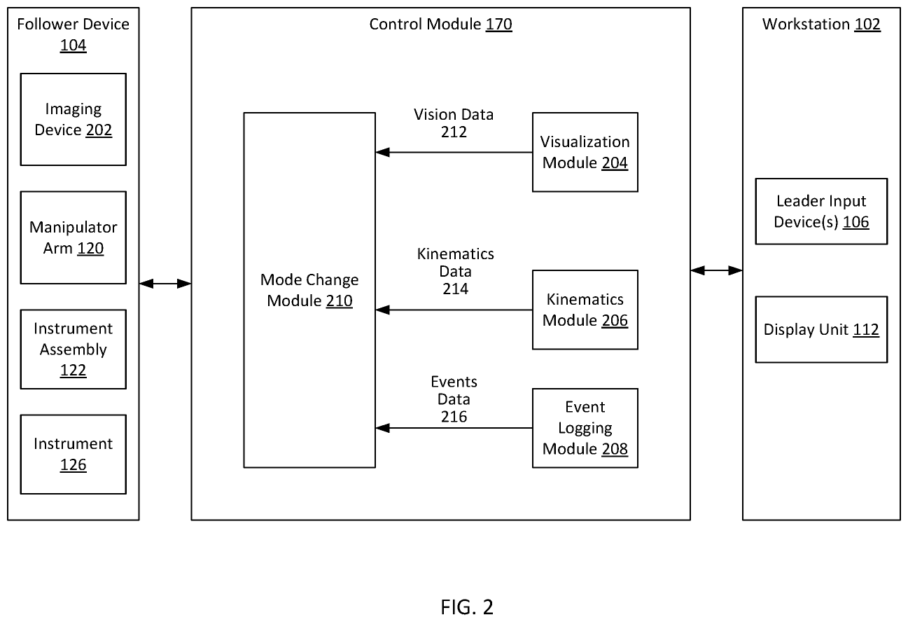

FIG. 2 illustrates control module 170 of FIG. 1 in greater detail, according to various embodiments. As shown, control module 170 includes, without limitation, a visualization module 204, a kinematics module 206, an event logging module 208, and a mode change module 210. Follower device 104, besides including manipulator arm 120, instrument assembly 122, and instrument 126 as described above with reference to FIG. 1, further includes imaging device 202. Workstation 102 includes leader input device(s) 106 and display unit 112, as described above with reference to FIG. 1.

Kinematics module 206 receives information of joint positions and/or velocities of joints in leader input device(s) 106. In some embodiments, the joint positions may be sampled at a control system processing rate. Kinematics module 206 processes the joint positions and velocities and transforms them from positions and velocities of a reference coordinate system associated with leader input device(s) 106 (e.g., a joint space of leader input device(s) 106) to corresponding positions and velocities of a reference coordinate system associated with follower device 104. In some embodiments, the reference coordinate system associated with follower device 104 is a coordinate system associated with imaging device 202 or a coordinate system with instrument 126 in a field of view of imaging device 202. In some examples, kinematics module 206 accomplishes this transformation in any technically feasible manner (e.g., using one or more kinematic models, homogeneous transforms, and/or the like). In some embodiments, the reference coordinate system of imaging device 202 may be a reference coordinate system for eyes of operator 108. In some embodiments, kinematics module 206 ensures that the motion of instrument 126 in a reference coordinate system of imaging device 202, corresponds to the motion of leader input device(s) 106 in the reference coordinate frame for the eyes of operator 108. In some embodiments, kinematics module 206 receives information of joint positions and/or velocities associated with follower device 104 (e.g., joint positions and/or velocities of joints in manipulator arm 120, position and/or orientation of instrument 126). Kinematics module 206 provides kinematics data 214 comprising these positions and/or velocities associated with follower device 104 to a mode change module 210.

In various embodiments, follower device 104 includes one or more imaging devices 202. Imaging device 202 can be an instrument 126 (e.g., an endoscope) that is included in an instrument assembly 122 and coupled to a manipulator arm 120. Additionally or alternatively, follower device 104 can include an imaging device 202 that is positioned to capture views of an instrument 126 (e.g., a view of the distal end of instrument 126 and any part of the worksite that is in proximity). In some embodiments, imaging device 202 is a monoscopic or stereoscopic camera, a still or video camera, an endoscope, a hyperspectral device, an infrared or ultrasonic device, an ultrasonic device, a fluoroscopic device, and/or the like. Images captured by one or more imaging device 202 can be processed by a visualization module 204 for display on display unit 112. Imaging device 202 can be single or multi-spectral, for example capturing image data in one or more of the visible, infrared, and/or ultraviolet spectrums. Visualization module 204 provides vision data 212 comprising these images captured by imaging device 202 to mode change module 210.

Event logging module 208 logs events in computer-assisted system 100. Event logging module 208 monitors computer-assisted system 100 and components thereof (e.g., obtain event information from workstation 102 and/or follower device 104, monitor transmissions within computer-assisted system 100, monitor one or more operational parameters associated with follower device 104), identifies events based on the monitoring, and logs the events in an event log, which can be stored in memory 160. Event logging module 208 provides events data 216 of events from the event log to mode change module 210.

Instrument 126 can perform different actions according to different modes or functionalities, and/or perform an action according to different approaches and/or parameters. In various embodiments, mode change module 210 can effect changes in mode, functionality, approach, and/or parameter for an instrument 126 based on gestures performed by operator 108 using instrument 126 during a procedure. Mode change module 210 can acquire, as inputs, data from kinematics module 206, visualization module 204, and/or event logging module 208 (e.g., vision data 212, kinematics data 214, and/or events data 216, respectively). Mode change module 210 processes the acquired input data, recognizes a gesture in a motion of instrument 126 based on the processing, and issues control signals to effect a change in the mode of the operation of instrument 126 based on the recognized gesture. Further details regarding mode change module 210 are described below.

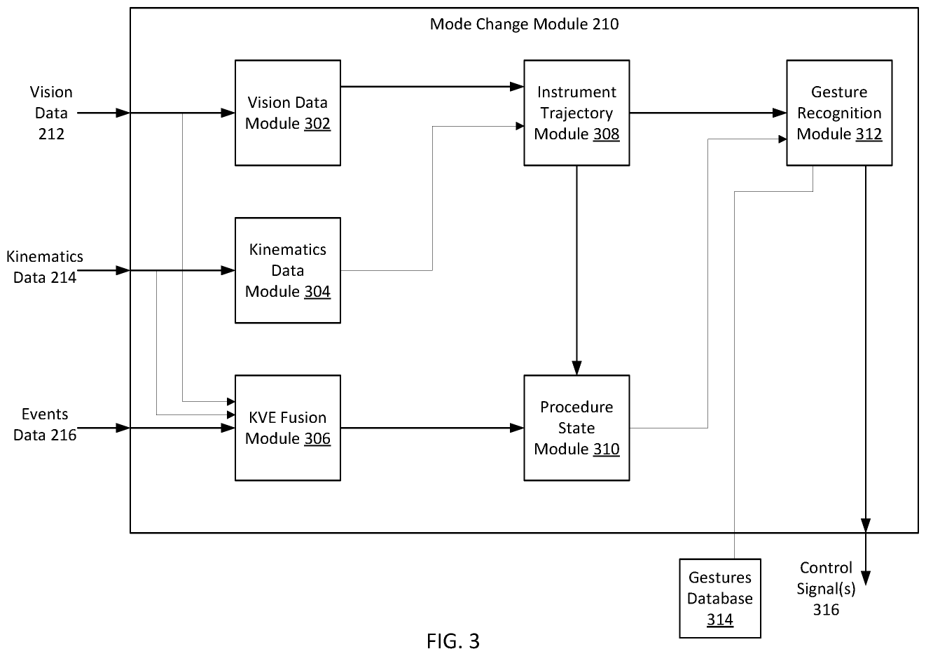

FIG. 3 illustrates mode change module 210 of FIG. 2 in greater detail, according to various embodiments. Mode change module 210 includes one or more modules configured to process vision data 212, kinematics data 214, and/or events data 216 to recognize a gesture performed using instrument 126 during a procedure. As shown, mode change module 210 includes, without limitation, vision data module 302, kinematics data module 304, kinematics/vision/event (KVE) fusion module 306, instrument trajectory module 308, procedure state module 310, and gesture recognition module 312. Mode change module 210 can access a gestures database 314 stored in memory 160. Mode change module 210 can output, without limitation, control signal(s) 316.

In various embodiments, mode change module 210 uses machine learning-based techniques to recognize a gesture performed using instrument 126 during a procedure. Mode change module 210 analyzes recent data associated with instrument 126 and with computer-assisted system 100 to determine a trajectory of instrument 126 and a state of a current procedure in which the trajectory occurs. Based on the trajectory of instrument 126 and the procedure state, mode change module 210 recognizes whether operator 108 has performed a gesture using instrument 126. After recognizing the gesture performed using with instrument 126, mode change module 210 can identify a change in a mode, functionality, approach, and/or parameter (collectively referred to as a “mode change” below for sake of brevity) for instrument 126 based on the recognized gesture, and issue signals, command, and/or the like (e.g., control signal(s) 316) to cause the mode change. The mode change can include, without limitation, changing an active mode or functionality of instrument 126 from one mode or functionality to another, changing a value of an operating parameter associated with instrument 126 (e.g., an amount of grip force, a sealing energy level), and/or the like.

Mode change module 210 receives as inputs vision data 212, kinematics data 214, and/or events data 216. Vision data 212 includes images (e.g., still images, video) captured by imaging device 202 (e.g., a stereoscopic or monoscopic endoscope) of follower device 104 during the procedure. For example, the images can include images captured, by imaging device 202, from a perspective of imaging device pointed in the same direction as instrument 126 (e.g., captured by an endoscope integrated with instrument 126) and/or from a third-person view relative to instrument 126. More generally, vision data 212 includes images that captures positions and orientations of instrument 126 over time, from which a movement of instrument 126 (e.g., movement of a distal portion of instrument 126) can be determined. In some embodiments, images can be captured at a certain frequency (e.g., at a frame rate of imaging device 202), and images can be sampled from the set of captured images at the capture frequency (e.g., sampling rate is the same as the frame rate of imaging device 202) or at a different frequency (e.g., sampling rate can be higher or lower than the frame rate). In some embodiments, the image sampling rate is a predefined rate (e.g., 10 frames per second). In some embodiments, mode change module 210 can acquire vision data 212 directly from imaging device 202, or indirectly via visualization module 204.

Kinematics data 214 includes data indicating the position, orientation, speed, velocity, pose, and/or shape of instrument 126 (e.g., of the distal end of instrument 126 in particular) and/or of one or more links, arms, joints, and/or the like of a kinematic structure supporting instrument 126 (e.g., manipulator arm 120). In some embodiments, kinematics data 214 can be sampled at the same sampling frequency as images are sampled from vision data 212. In some embodiments, mode change module 210 can acquire kinematics data 214 from kinematics module 206.

Events data 216 includes data indicating events logged by event logging module 208 in, for example, an events log associated with computer-assisted system 100 as a whole and/or with any component thereof. Non-limiting examples of events logged in events data 216 include instrument 126 contacting an object in the worksite, instrument 126 removing that contact, imaging device 202 being activated or deactivated, activation of an input device (e.g., pedal, lever, button, voice input, a graphical user interface on display unit 112) by operator 108, a current state of computer-assisted system 100, activation of an energy delivery mode for instrument 126, opening or closure of jaws on an end effector on instrument 126, selection of a mode by operator 108, and so forth.

In some embodiments, the kinematics data 214 and/or the events data 216 includes input from one or more input devices. For example, instrument 126 can be controlled using a touchpad as the input device with taps and/or other gestures for registering the input and sequencing the inputs. For example, a touchpad could be placed into a gesture mode where a first gesture is a first command for a sequence and a second gesture is a second command for the same sequence. For example, a first tap on the touchpad could command a needle throw, and a second tap on the touchpad could command that the needle be reloaded. In some examples, the touchpad includes one or more force and/or pressure sensors that provide an analog/variable input. In some examples, the touchpad includes one or more touch, force, pressure, and/or presence sensors that provide a binary on/off input.

In some embodiments, vision data 212, kinematics data 214, and/or events data 216 are synchronized and sub-sampled from respective original rates (e.g., an image capture rate, a kinematics data capture rate, an event logging rate, etc.) to a predefined rate (e.g., 10 Hz).

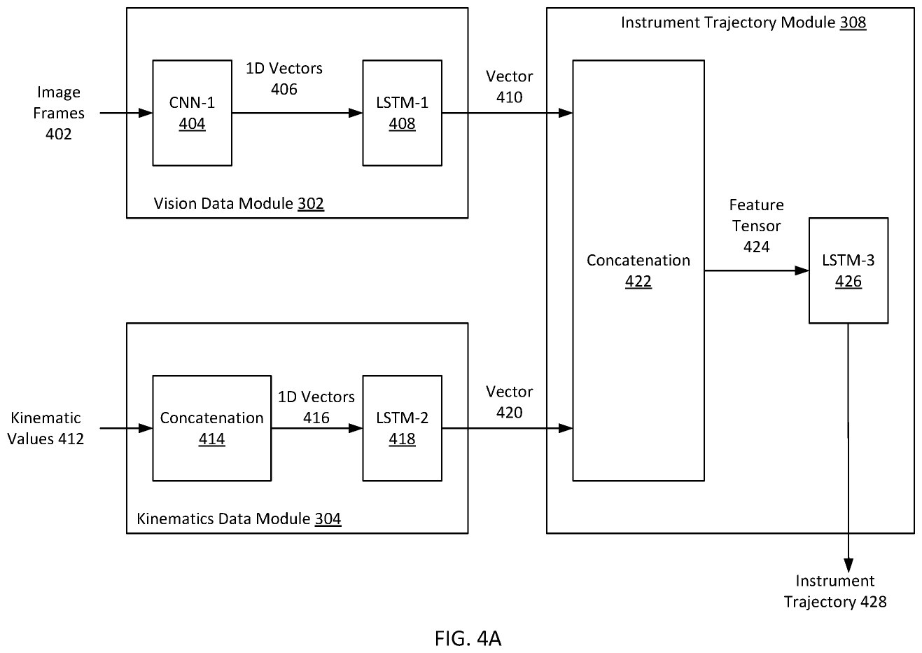

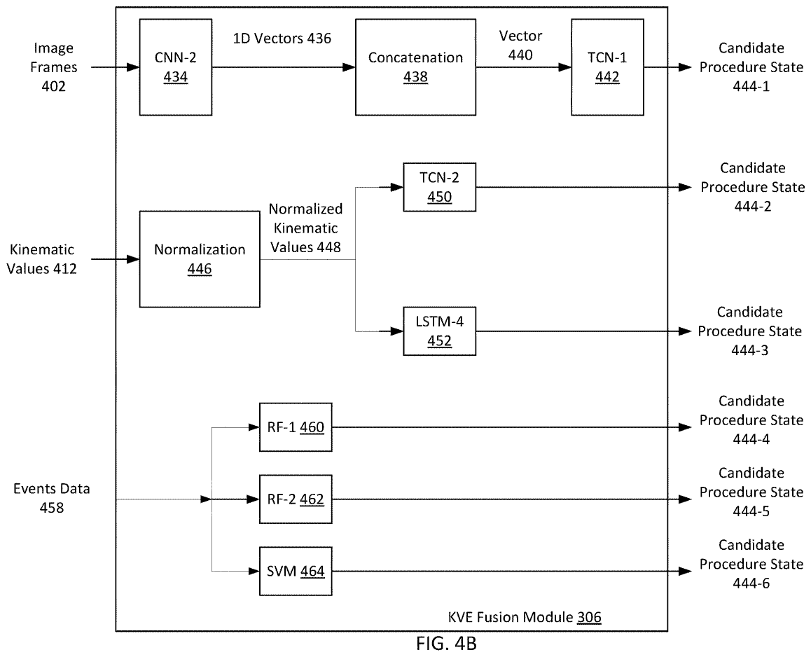

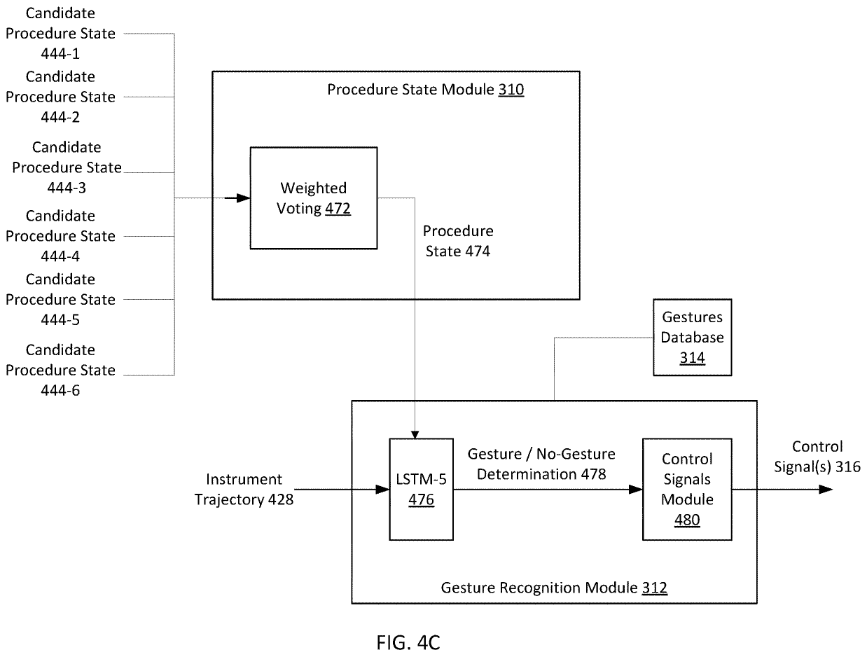

Vision data module 302 samples images from vision data 212 and processes the sampled images (e.g., the last 20 samples) to generate an output. In some embodiments, processing of vision data 212 by vision data module 302 includes generating data representations of the sampled images, and processing the data representations of the sampled images to analyze temporal relationships between the images. In some examples, vision data module 302 outputs an intermediate output (e.g., a vector) based on the analysis for processing by other modules within mode change module 210. In some embodiments, vision data module 302 generates the data representations, analyze the data representations, and output the intermediate output using one or more machine learning techniques (e.g., neural networks and models), an example of which is described below in conjunction with FIGS. 4A-4C.

In some embodiments, vision data 212 data includes left and right 3D images from a stereoscopic imaging device, 3D images from multiple imaging devices setup to capture images from different perspectives, and/or 3D depth or intensity maps from the imaging device. In some embodiments, vision data module 302 can process a stereoscopic image frame to generate a data representation of the stereoscopic image frame by generating a one-dimensional (1D) vector representation of at least one “eye” of the stereoscopic image frame (e.g., the left and/or the right 3D image). In some embodiments, vision data module 302 also generates a data representation (e.g., 1D vector) of a region of interest around a distal end of instrument 126 as captured in the sampled image. More generally, for a given image, vision data module 302 generates one or more data representations for analysis, where each of multiple data representations for a given image can be directed to different aspects of the given image (e.g., a left or right 3D image, a region of interest around the distal end of instrument 126 as captured in the image).

Kinematics data module 304 samples kinematics data 214 and processes the sampled kinematics data 214. In some embodiments, processing of kinematics data 214 by kinematics data module 304 includes generating data representations of the kinematics data, and analyzing the data representations of the kinematics data to generate an intermediate output. Kinematics data module 304 outputs an intermediate output (e.g., a vector) for processing by other modules within mode change module 210. In some embodiments, kinematics data module 304 generates the data representations, analyzes the data representations, and outputs the intermediate output using one or more machine learning techniques (e.g., neural networks and models), an example of which is described below in conjunction with FIGS. 4A-4C.

In some embodiments, kinematics data module 304 generates the data representation for a given sampling time point by concatenating the kinematics data (e.g., angles, positions, velocities) for one or more links, joints, arms, or the like, corresponding to the same timestamp as a sampled image, into a 1D vector.

Instrument trajectory module 308 processes and analyzes intermediate outputs generated by vision data module 302 and/or kinematics data module 304 to determine a trajectory of instrument 126 (e.g., a trajectory or path of the distal end of instrument 126). That is, instrument trajectory module 308 determines an instrument trajectory using data representing images of instrument 126 and/or data representing positions, orientations, velocities, etc. of joints and/or the like associated with follower device 104. The determined trajectory can be represented using any suitable data representation (e.g., a vector, a matrix, etc.). In some embodiments, the determined trajectory is associated with a confidence level of the determination. In some embodiments, instrument trajectory module 308 can determine multiple candidate trajectories, with different confidence levels.

KVE fusion module 306 acquires and processes vision data 212 and kinematics data 214 in a similar manner as vision data module 302 and kinematics data module 304, respectively. That is, KVE fusion module 306 samples image data and kinematics data, generate data representations of the sampled image data and kinematics data, and analyze the data representations to generate one or more intermediate outputs. In some embodiments, KVE fusion module 306 samples data at the same rate or at a different rate than vision data module 302 and/or kinematics data module 304.

KVE fusion module 306 samples images from vision data 212 and generates data representations (e.g., 1D vector representations) of the sampled images (e.g., the last 32 samples). KVE fusion module 306 then combines the data representations of the sampled images into a larger data representation (e.g., a larger 1D vector) and analyzes the larger data representation to generate an intermediate output.

KVE fusion module 306 samples kinematics data 214 and generates data representations (e.g., 1D vector representations) of the sampled kinematics data 214. KVE fusion module 306 then analyzes the kinematics data representations using one or more techniques to generate one or more intermediate outputs.

KVE fusion module 306 also samples events data 216 and processes the sampled events data using one or more techniques (e.g., classification models) to generates data representations (e.g., 1D vector representations) of the sampled events data 216. KVE fusion module 306 then analyzes the events data representations using one or more techniques to generate one or more intermediate outputs.

In some embodiments, an intermediate output generated by KVE fusion module 306 includes a state of the current procedure, as determined based on vision data 212, kinematics data 214, or events data 216 using one or more techniques. In some examples, the intermediate output includes a sequence (e.g., as in a timeline) of procedure states. That is, the intermediate output can be a sequence of procedure states ordered by time.

A procedure state module 310 receives the intermediate outputs generated by KVE fusion module 306 and processes the intermediate outputs using any suitable technique (e.g., weighted or unweighted voting) to determine a state of the procedure contemporary with the instrument trajectory. The procedure state is a determination of a state of the procedure when the instrument trajectory occurred. In some embodiments, the determined procedure state is a sequence of procedure states. An example of a sequence of procedure states can be operator 108 lifting an identified instrument 126, then moving instrument 126 to a surface in the worksite, and then sweeping instrument 126 over the surface. In some embodiments, the determined procedure state is associated with a confidence level of the determination. In some embodiments, KVE fusion module 306 determines multiple candidate procedure states, with different confidence levels, and procedure state module 310 selects a candidate procedure state with the highest confidence level as procedure state 474. Additionally or alternatively, in some embodiments, procedure state module 310 combines the candidate procedure states to determine an aggregated or combined procedure state 474.

Gesture recognition module 312 receives a determined trajectory of instrument 126 from instrument trajectory module 308, and a determined procedure state from procedure state module 310. Gesture recognition module 312 analyzes the determined trajectory of instrument 126 and the determined procedure state to recognize a gesture. In some embodiments, the gesture recognition includes determining one or more gestures in a gestures database 314 that match the determined instrument trajectory and the determined procedure state, or determine that no gesture in gestures database 314 has occurred. In some embodiments, a determination of a gesture or no-gesture (e.g., a match in gestures database 314) is associated with a confidence level of the determination. For example, gesture recognition module 312 can determine multiple candidate gestures matches in gestures database 314 and/or no-gesture, each with a confidence level. In some embodiments, gestures database 314 is a database, or more generally any suitable data repository or structure (e.g., a table), that is stored in memory 160, and can be structured in any suitable manner. Gestures database 314 includes a database of gestures, corresponding instrument trajectories and procedure states, and corresponding control signals.

Based on the determined gesture or no gesture, gesture recognition module 312 generates corresponding control signal(s) 316 or takes no action, respectively. If the determined gesture is “no gesture” (e.g., “no gesture” is the determined candidate with a confidence level that meets a minimum threshold and is the highest amongst the candidates), then gesture recognition module 312 would disregard the trajectory and procedure state. That is, mode change module 210 takes no action (e.g., no mode change to take place) with respect to the determined instrument trajectory and procedure state. Mode change module 210 continues to sample and process vision data 212, kinematics data 214, and/or events data 216 to determine an updated instrument trajectory and procedure state, and to recognize a gesture based on the updated instrument trajectory and procedure state. In some embodiments, mode change module 210 continuously or periodically generates and updates the instrument trajectory and procedure state by sampling and processing vision data 212, kinematics data 214, and/or events data 216 using a rolling time window or a rolling window of samples.

If gesture recognition module 312 determines a gesture (e.g., a gesture from gestures database 314 is the candidate with a confidence level that meets a minimum threshold and is the highest amongst the candidates), then gesture recognition module 312 generates and transmits corresponding control signal(s) 316. Gesture recognition module 312 retrieves definitions or specifications of the corresponding control signal(s) 316 from gestures database 314, generates the signals, and outputs the signals for transmission to follower device 104. The control signal definition or specification identifies the associated mode change and specifies the control signal(s) that commands follower device 104 to effect the mode change. Follower device 104, in response to receiving control signal(s) 316, changes a mode of instrument 126 based on the received control signal(s) 316. Mode change module 210 can continue to sample and process vision data 212, kinematics data 214, and/or events data 216 to determine an updated instrument trajectory and procedure state, and to recognize a gesture based on the updated instrument trajectory and procedure state. In some embodiments, mode change module 210 continuously or periodically generates and updates the instrument trajectory and procedure state by sampling and processing vision data 212, kinematics data 214, and/or events data 216 using a rolling time window or a rolling window of samples.

In some embodiments, mode change module 210 and/or control module 170 prompt operator 108 for confirmation and/or additional information associated with a mode change, before transmitting control signals(s) 316 to follower device 104. For example, mode change module 210 and/or control module 170 can prompt operator 108 for confirmation of the mode change (e.g., via a voice prompt, via a prompt displayed in a user interface on display unit 112). Mode change module 210 and/or control module 170 can also prompt operator 108 for additional information (e.g., a parameter value) associated with the mode change. Operator 108 can provide an input to respond to the prompt using any input method suitable for computer-assisted system 100 (e.g., input button, foot pedal, touch screen input, voice input, performing a hand gesture, performing a gesture with instrument 126).

In some embodiments, instrument trajectory module 308 and/or procedure state module 310 are combined with gesture recognition module 312. That is, gesture recognition module 312 performs the functionality of instrument trajectory module 308 and/or procedure state module 310 described above, as well as the functionality of gesture recognition module 312 described above.

In operation, during a procedure, mode change module 210 samples vision data 212, kinematics data 214, and/or events data 216, and processes the sampled data to recognize a gesture performed using instrument 126 or determine that no gesture has occurred. If mode change module 210 determines that no gesture has occurred, then mode change module 210 would take no action with regard to changing a mode of the instrument, and then samples further data to make an updated determination. If mode change module 210 recognizes a gesture, then mode change module 210 would generate and output control signals 316 associated with the recognized gesture to effect the mode change.

FIGS. 4A-4C illustrates an example machine learning implementation of mode change module 210, according to some embodiments. As described above, mode change module 210 uses one or more machine learning-based techniques. Examples of machine learning techniques that mode change module 210 can implement include but are not limited to neural networks, convolutional neural networks (CNN), long short-term memories (LSTM), temporal convolutional networks (TCN), random forests (RF), support vector machines (SVM), and/or the like as well as associated models. While FIGS. 4A-4C illustrate a specific machine learning implementation of mode change module 210, it should be appreciated that other machine learning implementations, or combinations thereof, are possible.

FIG. 4A illustrates vision data module 302, kinematics data module 304, and instrument trajectory module 308 in further detail. Vision data module 302 as shown uses a CNN-1 404 and an LSTM-1 408 to process vision data 212. CNN-1 404 receives sampled image frames 402 from vision data 212 as input. CNN-1 404 processes image frames 402 to recognize instrument 126 within the image frames. CNN-1 404 outputs 1D vector representations 406 of image frames 402. 1D vector representations 406 represents recognition of instrument 126, and positions thereof, in images captured by imaging device 202 within a time window. In some embodiments, CNN-1 404 is a visual geometry group (VGG) convolutional neural network (e.g., VGG-16 with 16 convolutional layers).

LSTM-1 408 receives 1D vector representations 406 as input. LSTM-1 408 processes 1D vector representations 406 of image frames 402 with persistence or memory of prior image frames. Accordingly, LSTM-1 408 can track a position, and correspondingly movement, of instrument 126 or portions of instrument 126 within image frames 402. LSTM-1 408 outputs a vector 410 that represents the temporal relationships between image frames 402.

Kinematics data module 304 as shown uses an LSTM to process kinematics data 214. Kinematics data module 304 concatenates 414 sampled kinematics values 412 from kinematics data 214 into 1D vector representations 416. In some embodiments, kinematics values 412 are normalized onto a predefined scale before concatenation. LSTM-2 418 receives 1D vector representations 416 as input. LSTM-2 418 processes 1D vector representations 416 of kinematics values 412 with persistence or memory of prior kinematics values. LSTM-2 418 outputs a vector 420. In some embodiments, LSTM-2 418 is an attention-based LSTM.

Instrument trajectory module 308 concatenation module 422 vector 410 and vector 420 into a feature tensor 424. LSTM-3 426 implemented within instrument trajectory module 308 receives feature tensor 424 as an input. In some embodiments, concatenation module 422 concatenates, for a given sampling time point, the vector output by vision data module 302 and the vector output by kinematics data module 304 into a single feature tensor 424 for the sampling time point. LSTM-3 426 processes feature tensor 424, with persistence or memory of prior feature tensors, to determine an instrument trajectory 428. In some embodiments, LSTM-3 426 also determines a confidence level associated with instrument trajectory 428. In some embodiments, LSTM-3 426 determines multiple candidate instrument trajectories 428 with respective confidence levels. In some embodiments, LSTM-3 426 is an attention-based LSTM.

FIG. 4B illustrates KVE fusion module 306 in further detail. KVE fusion module 306 can implement one or more CNNs, one or more TCNs, one or more LSTMs, one or more RFs, and one or more SVMs. CNN-2 434 receives sampled image frames 402 as input. CNN-2 434 processes image frames 402 to recognize instrument 126 or portions of instrument 126 within image frames 402, outputting 1D vector representations 436. In some embodiments, CNN-2 434 is a VGG (e.g., VGG 16) convolutional neural network. KVE fusion module 306 includes a concatenation module 438 that concatenates 1D vector representations 436 into a vector 440. TCN-1 442 receives vector 440 as input. TCN-1 442, analyzes vector 440, corresponding to image frames 402 over time, with causal convolution to generate a first candidate procedure state 444-1 as output. Candidate procedure state 444-1 is a determination (e.g., a prediction, a classification) of the procedure state by TCN-1 442 based on vector 440. In some embodiments, TCN-1 442 also outputs a confidence level associated with candidate procedure state 444-1.

KVE fusion module 306 normalizes 446 sampled kinematics values 412 into normalized kinematics values 448, which in some embodiments can be represented as a 1D vector. Each of TCN-2 450 and LSTM-4 452 receives normalized kinematics values 448 as input. TCN-2 450 analyzes normalized kinematics values 448 with causal convolution, outputting a candidate procedure state 444-2. Candidate procedure state 444-2 is a determination (e.g., a prediction) of the procedure state by TCN-2 450 based on normalized kinematics values 448. LSTM-4 452 processes normalized kinematics values 448 with persistence or memory of prior kinematics values, outputting a candidate procedure state 444-3. Candidate procedure state 444-3 is a determination (e.g., a prediction, a classification) of the procedure state by LSTM-4 452 based on normalized kinematics values 448. In some embodiments, TCN-2 450 and LSTM-4 452 also outputs a confidence level associated with candidate procedure states 444-2 and 444-3, respectively.

Each of classification models RF-1 460, RF-2 462, and SVM 464 receives sampled events data 458 from events data 216 as input. RF-1 460 and RF-2 462 respectively process sampled events data 458 to classify sampled events data 458 into a candidate procedure state. Both RF-1 460 and RF-2 462 analyze sampled events data 458 via a set of random decision trees, with a difference between RF-1 460 and RF-2 462 being a different number of trees (e.g., 400 trees and 500 trees respectively). SVM 464 also analyzes sampled events data 458 to classify sampled events data 458 into a candidate procedure state. RF-1 460, RF-2 462, and SVM 464 outputs candidate procedure states 444-4, 444-5, and 444-6, respectively.

FIG. 4C illustrates procedure state module 310 and gesture recognition module 312 in further detail. Continuing in FIG. 4C, procedure state module 310 receives candidate procedure states 444-1 thru 444-6 as inputs. Procedure state module 310 analyzes candidate procedure states 444-1 thru 444-6 using a weighted voting technique 472 to determine (e.g., select a candidate procedure state 444, combine candidate procedure states 444) a procedure state 474. In some embodiments, weighted voting technique 472 applies weighted voting to the confidence levels of candidate procedure states 444, i.e., votes based on the confidence levels of candidate procedure states 444.

Gesture recognition module 312 receives procedure state 474 and instrument trajectory 428 as inputs. LSTM-5 476, implemented in gesture recognition module 312, analyzes procedure state 474 and instrument trajectory 428 together, with persistence or memory of prior procedure states and instrument movement, to determine whether procedure state 474 and instrument trajectory 428 matches a gesture in gestures database 314. In some embodiments, LSTM-5 476 classifies procedure state 474 and instrument trajectory 428 into one or more matching gestures in gestures database 314 with respective confidence levels. LSTM-5 476 can also make a no-gesture determination based on procedure state 474 and instrument trajectory 428 (e.g., if no match in gestures database 314 meets a minimum confidence level threshold). LSTM-5 476 outputs a gesture/no-gesture determination 478 indicating a matching gesture or no-gesture determination.

A control signals module 480 receives gesture/no-gesture determination 478. If gesture/no-gesture determination 478 indicates no gesture, then control signals module 480 can indicate that no mode change is to take place. If gesture/no-gesture determination 478 includes a matching gesture from gestures database 314, then controls signals module 4870 would retrieve control signal specifications associated with the matching gesture from gestures database 314 and generate corresponding control signals 316.

In some embodiments, machine learning networks and models (e.g., neural networks, etc.) in mode change module 210 are trained in an order. For example, networks in KVM fusion module 306 (e.g., CNN-2 434, TCN-1 442, TCN-2 450, LSTM-4 452, RF-1 460, RF-2 462, and SVM 464) are trained first. These networks are trained to determine (e.g., classify, predict) a procedure state) based on training data sets corresponding to vision data 212, kinematics data 214, and/or events data 216. Those networks, after being trained, are frozen. Then, the other machine learning networks (e.g., CNN-1 404, LSTM-1 408, LSTM-2 418, LSTM-3 426, LSTM-5 476) in mode change module 210 are trained in conjunction with the frozen machine learning networks, also using training data sets corresponding to vision data 212, kinematics data 214, and/or events data 216.

In some embodiments, training is implemented by minimizing a categorical cross entropy between the prediction and ground truth data. Training data is acquired by preforming gestures alongside normal instrument motion during procedures, using a logging application to collect synchronized images, kinematics, and/or events. Ground truth data can be manually annotated by humans to indicate the states, gestures, etc. corresponding to the ground truth data.

In some embodiments, gestures are predefined. That is, a set of gestures (e.g., the corresponding instrument trajectories and procedure states) are predefined before implementation of mode change module 210, and mode change module 210 is trained to recognize the predefined gestures. In some embodiments, gestures are defined post-implementation (e.g., user-defined), and mode change module 210 is re-trained to recognize the post-implementation defined gesture as well as the predefined gestures.

In some embodiments, gestures that are defined for recognition by mode change module 210 preferably are ones that are less susceptible to false positives (low false positive rate), false negatives (low false negative rates), and confusion with other gestures (low chance of misclassification as a different gesture). Additionally, in some embodiments, gestures that can be recognized with low lag times are preferrable.

In some embodiments, gestures defined for recognition by mode change module 210 include active and passive gestures. As used herein, an active gesture is a gesture that includes an instrument motion that is not a part of a task associated with a procedure. That is, the instrument motion does not flow within the task naturally and/or is a significant deviation from the task. In some embodiments, an active gesture includes an instrument motion that is distinct from movement of the instrument that occurs during execution of a procedure being performed using the instrument. Active gestures can be predefined before implementation or defined post-implementation.

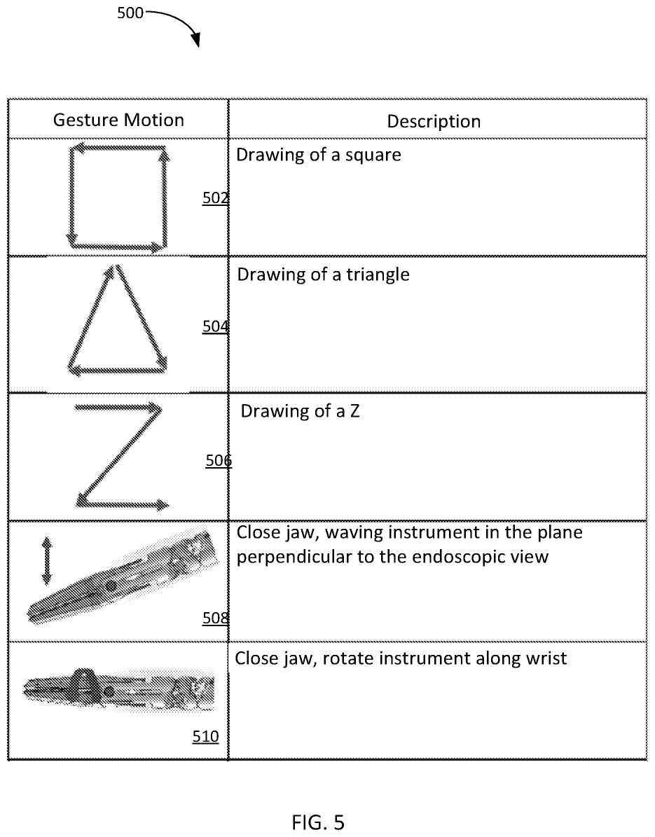

As used herein, a passive gesture is a gesture that includes an instrument motion that can be a part of a task associated with a procedure. That is, the instrument motion flows within the task naturally and/or is at most a trivial or negligible deviation from the task. In some embodiments, a passive gesture includes an instrument motion that occurs during execution of a task as part of a procedure being performed using the instrument. Passive gestures can also be predefined before implementation or defined post-implementation. In some embodiments, whether a gesture for a mode/functionality change should be defined as an active gesture or passive gesture can depend on how disruptive to the flow the mode/functionality change and the gesture would be during an associated procedure. Examples of active and passive gestures are described below in conjunction with FIG. 5. Definitions of active and passive gestures, and corresponding mode/functionality changes and events, are stored in gestures database 314.

In some embodiments, gestures database 314 includes various types of mode/functionality changes for various types of instruments. In some examples, gestures database 314 includes pairs of modes, where the same instrument motion can toggle between a pair of modes, or more generally between a set of two or more modes, depending on the state of the procedure. Alternatively, different instrument motions correspond to the respective modes in a set of modes. That is, one instrument motion is associated with one mode, and a different instrument motion is associated with another mode. Other gestures included in gestures database 314 are gestures that signal to control system 140 that an operator wants to adjust a parameter or other behavior in computer-assisted system 100 (e.g., in follower device 104 in particular). Table 1 below illustrates examples of pairings of modes, and parameter or behavior adjustment changes that can be mapped to gestures. It should be appreciated, however, that the pairs of modes and adjustable parameters and adjustments below are merely exemplary, and more or less changeable modes, functions, parameters, and behaviors are possible.

TABLE 1
Between Energy Modes

| | |
|---|---|
| Bipolar Seal | Monopolar Tip |
| Sealing Mode | Bipolar Only |
| Increase Voltage | Decrease Voltage |
| Radio Frequency Cut Sine | Radio Frequency Cut Square Wave |
| Wave | |
| Ultrasonic Seal | Ultrasonic Cut |

Between Cut/Seal Modes

| | |
|---|---|
| Single-Step Seal and Cut | Sequential Seal then Cut |
| Turn Off Sealing | Turn On Sealing |
| Standard Cut | Extended Cut |
| Sealing Mode | Bipolar Cut at Tip |
| Sealing Mode | “Wrapping Paper” Cut (partially open |
| | jaws with blade extended) |

Between Suction and Irrigation Modes

| | |
|---|---|
| Suction Mode | Irrigation Mode |

Between Task-Based Modes

| | |
|---|---|
| Sealing Mode | Needle-driving Mode |

Other Adjustable Parameters or Behaviors
Increase instrument range of motion (ROM)
Change behavior of leader assembly
Change in grip force
Change imaging modes
Take a screenshot

FIG. 5 is a table 500 illustrating example active and passive gestures according to some embodiments. In table 500, gestures 502, 504, and 506 are active gestures, and gestures 508 and 510 are passive gestures.

Gesture 502 includes an operator drawing a square using instrument 126. That is, operator manipulates a leader device that causes instrument 126 to draw a square shape using the distal end of instrument 126 during the procedure. Mode change module 210 recognizes the trajectory of instrument 126 as drawing a square, and maps the associated gesture to a mode change. Similarly, gesture 504 includes drawing a triangle, and gesture 506 includes drawing a letter Z. Other examples of active gestures include drawing other geometrical shapes (e.g., rectangle), alphabet letters, and/or numbers.

Gesture 508 includes the operator closing a jaw of an instrument 126 that includes a gripping jaw, and then waving instrument 126 in a plane perpendicular to the current view of an imaging device. Gesture 510 includes the operator closing a jaw of an instrument 126 that includes a gripping jaw, and then rotating instrument 126 along the wrist. Other examples of passive gestures include reaching for and/or grasping a needle during a procedure that would expectedly include usage of the needle.

Specific examples of recognition of active and passive gestures for illustration purposes will now be described. The examples described below both involve a medical context. It should be appreciated that these specific examples are merely exemplary and not intended to be limiting.

In a first example, operator 108 (e.g., a surgeon) is controlling an instrument that includes a simultaneous seal-and-cut mode and a sequential seal-then-cut mode. Operator 108 is attempting to seal and cut through fatty or connective tissue with low vascularity (e.g., omentum, mesentery, etc.) to get to a target anatomy. Operator 108 wants to move quickly and efficiently through this tissue and the risk of bleeding is low, so a simultaneous seal-and-cut mode is used for this portion of the procedure. Operator 108 then comes to tissue with high vascularity or to a large important vessel (e.g., inferior mesenteric artery during a colectomy procedure), where operator 108 wants to move cautiously because the bleeding risk is high and the anatomy is critical. Operator 108 may want to perform double seals or use a banding technique on a large vessel before ultimately cutting. Operator 108 can perform an active gesture (e.g., draw a square with the instrument) to mode-switch into a sequential seal-then-cut mode, giving operator 108 the more precise and careful control desired for this portion of the procedure without having to open or switch to a different instrument or otherwise significantly break surgical flow. When operator 108 moves back to less critical tissue with lower vascularity or to a portion of the procedure where operator 108 wants to move quickly, operator 108 can mode switch back into simultaneous seal-and-cut mode, such as by using the active gesture used to mode switch into the sequential cut-then-seal more or a different active gesture. Operator 108 can switch back and forth between simultaneous and sequential seal and cut modes in numerous instances during a single procedure based on factors like tissue type, vascularity, criticality of the anatomy, difficulty of task, etc.

In a second example, operator 108 (e.g., a surgeon) has arrived at a suturing task during a procedure (e.g., closure of gastrostomy enterotomy defects during gastric bypass, installing mesh during hernia procedure, etc.). When the instrument grasps a needle, a mode switch is triggered to increase grip force to make needle driving easier and more effective. The presence of a needle in the instrument jaws acts as a passive gesture to activate the increase in grip force. The grip force can return to standard levels when the needle is no longer grasped by the instrument jaws; the release of the needle by the jaws is recognized as a passive gesture to return the grip force to standard levels. This allows variations in grip force to optimize the surgical task without having to swap instruments.

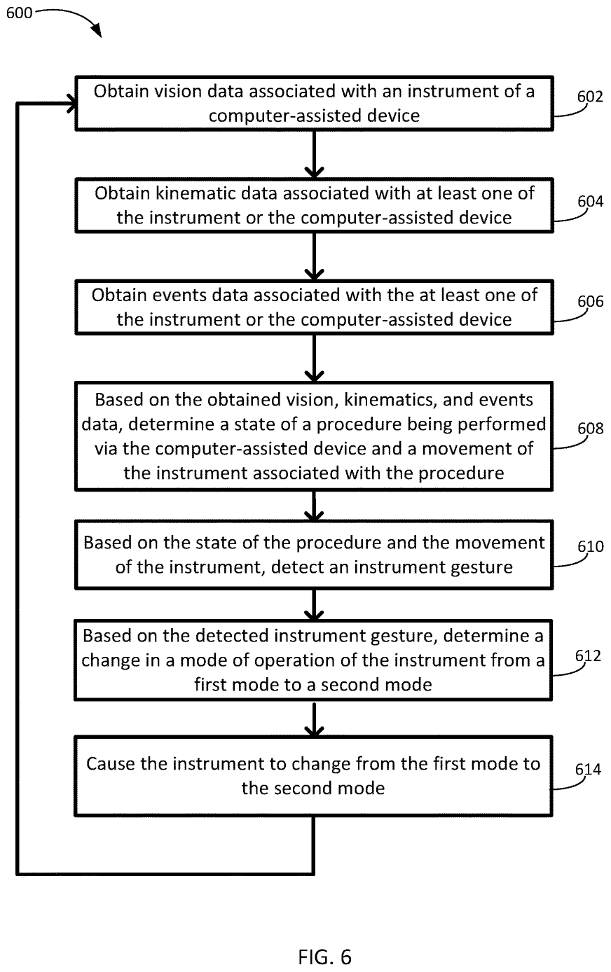

FIG. 6 is a flow chart of method steps for modifying the operation of an instrument, according to some embodiments. Although the method steps are described with respect to the systems of FIGS. 1-4, persons skilled in the art will understand that any system configured to perform the method steps, in any order, falls within the scope of the various embodiments. In some embodiments, one or more of the steps 602-614 of method 600 may be implemented, at least in part, in the form of executable code stored on one or more non-transient, tangible, machine readable media that when run by one or more processors (e.g., one or more processors of control system 140) cause the one or more processors to perform one or more of the steps 602-614. In some embodiments, portions of method 600 are performed by control module 170 and/or mode change module 210.

As shown, method 600 begins at step 602, where vision data associated with an instrument of a computer-assisted device is obtained. In some examples, the vision data includes one or more images of the instrument, such as instrument 126, captured by an imaging device, such as imaging device 202, located in a workspace. In some examples, the one or more images include monoscopic images, stereoscopic images, and/or combinations of the two. In some examples, the one or more images include images showing positions and/or orientations of the instrument over time. In some examples, the one or more images are captured from a perspective of the instrument, include a region of interest around the instrument, and/or the like. In some examples, the one or more images are processed by a visualization module, such as visualization module 204, to generate the vision data, such as vision data 212. In some examples, the vision data includes information related to the temporal relationships between the one or more images.

At step 604, kinematics data associated with at least one of the instrument or the computer-assisted device is obtained. The kinematics data includes information indicative of the position, orientation, speed, velocity, pose, shape, and/or the like related to the instrument, such as information about one or more joints, links, arms, and/or the like of a structure supporting the instrument. In some examples, the kinematics data includes one or more intermediate values generated by a kinematics module, such as kinematics module 206. In some examples, the kinematics data is synchronized with the vision data obtained during step 602.

At step 606, events data associated with at least one of the instrument or the computer-assisted device is obtained. The events data includes information indicative of events associated with computer-assisted system 100 and/or instrument 126 (e.g., state of instrument 126, events occurring at computer-assisted system 100, etc.). In some embodiments, events data includes events logged by a logging module, such as event logging module 208. In some embodiments, the events data is synchronized with the vision data and/or the kinematics data obtained in steps 602 and 604, respectively.

At step 608, based on the obtained vision, kinematics, and/or events data, a state of a procedure being performed via the computer-assisted device and a movement of the instrument associated with the procedure are determined. The vision data and/or the kinematics data can be processed to determine a movement trajectory of the instrument (e.g., instrument trajectory 428). In an example, the vision data and/or the kinematics data are respectively processed by one or more machine learning techniques to generate data representations of the data. The data representations are combined, and the combination is analyzed by one or more additional machine learning techniques to determine the instrument movement trajectory. Similarly, the vision data, kinematics data, and/or events data are also processed to determine a state of a current procedure being performed. In an example, the vision data, kinematics data, and events data, and/or data representations thereof, are respectively processed by a plurality of machine learning techniques to generate respective candidate procedure states, and a procedure state is determined from the plurality of candidate procedure states. In some embodiments, the one or more machine learning techniques and/or the one or more additional machine learning techniques are consistent with those discussed above with respect to FIGS. 4A-4C.

At step 610, based on the state of the procedure and the movement of the instrument, an instrument gesture is detected. The procedure state and the instrument movement trajectory, determined in step 608, are processed (e.g., by gesture recognition module 312) to recognize a gesture performed with the instrument or that no gesture has been performed. In an example, the procedure state and the instrument movement trajectory are processed by one or more machine learning techniques to match the procedure state and instrument trajectory to one or more candidate gestures in a gestures database (e.g., gestures database 314) and/or to a no-gesture determination. In some examples, the one or more candidate gestures and/or the no-gesture determination are also determined with respective confidence levels. A candidate gesture from the one or candidate gestures and/or a no-gesture determination is selected based on the confidence levels.

At step 612, based on the detected instrument gesture, determine a change in a mode of operation of the instrument from a first mode to a second mode. If a candidate gesture is selected in step 610, the corresponding mode change and associated control signal specification are identified and/or retrieved from the gestures database.

At step 614, mode change module 210 causes the instrument to change from the first mode to the second mode. One or more control signals (e.g., control signals 316) are generated (e.g., by control signals module 480) based on the control signal specification retrieved in step 612, and the one or more control signals are provided to affect the mode change.

In sum, a computer-assisted system for an instrument can change a mode or functionality of the instrument based on gestures performed during a procedure. Gestures include instrument motions performed by the operator during certain states in the procedure. The computer-assisted system can acquire vision data, kinematics data, and/or events data associated with the instrument and/or the computer-assisted system. The computer-assisted system can process the vision data, kinematics data, and/or events data to predict the state of the procedure and the instrument trajectory using machine learning techniques. The computer-assisted system can determine whether a gesture has occurred or not based on the predictions of procedure state and instrument trajectory. The computer-assisted system identifies the mode or functionality change corresponding to a recognized gesture and effect the mode or functionality change (e.g., transmit control signals to cause the change).

At least one advantage and technical improvement of the disclosed techniques relative to the prior art is that, with the disclosed techniques, the mode or functionality of an instrument can be changed without significant diversion from an on-going procedure.

Accordingly, the operator can maintain a high situational awareness with respect to the on-going procedure. Another advantage and technical improvement is that new instruments with multiple modes and/or functions can be added to a computer-assisted device without significant operator-facing modifications to the computer-assisted device or the user interface. Accordingly, a computer-assisted device can be expanded to include new instruments transparently and without a significant learning curve for the operator. These technical advantages provide one or more technological advancements over prior art approaches.

Any and all combinations of any of the claim elements recited in any of the claims and/or any elements described in this application, in any fashion, fall within the contemplated scope of the present disclosure and protection.

The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments.

Aspects of the present embodiments may be embodied as a system, method or computer program product. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “module,” a “system,” or a “computer.” In addition, any hardware and/or software technique, process, function, component, engine, module, or system described in the present disclosure may be implemented as a circuit or set of circuits. Furthermore, aspects of the present disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Aspects of the present disclosure are described above with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine. The instructions, when executed via the processor of the computer or other programmable data processing apparatus, enable the implementation of the functions/acts specified in the flowchart and/or block diagram block or blocks. Such processors may be, without limitation, general purpose processors, special-purpose processors, application-specific processors, or field-programmable gate arrays.

The flowchart and block diagrams in the figures illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

While the preceding is directed to embodiments of the present disclosure, other and further embodiments of the disclosure may be devised without departing from the basic scope thereof, and the scope thereof is determined by the claims that follow.

1. A computer-assisted device, the device comprising:
a structure configured to support an instrument for performing tasks at a worksite in response to manipulation of a leader input device by an operator;
memory storing an application; and
a processing system that, when executing the application, is configured to:
obtain kinematics data associated with at least one of the structure or the instrument;
based on at least the kinematics data recognize a gesture performed via the instrument; and
in response to recognizing the gesture, cause the computer-assisted device to change from a first mode of operation to a second mode of operation.

2. The device of claim 1, wherein the processing system is further configured to:
obtain vision data associated with the instrument; and
recognize the gesture based further on the vision data.

3. The device of claim 2, wherein the vision data comprises at least one of: one or more images capturing a perspective of the instrument, one or more images capturing the instrument, or one or more images capturing a region of interest in proximity to the instrument.

4. The device of claim 1, wherein the kinematics data comprises at least one of a position, an angle, an orientation, a speed, or a velocity of at least one of the structure or the instrument.

5. The device of claim 1, wherein the kinematics data is associated with at least a link, a joint, or an arm of the structure.

6. The device of claim 1, wherein the processing system is further configured to:
obtain events data associated with a least one of the structure or the instrument; and
recognize the gesture further based on the events data.

7. (canceled)

8. The device of claim 1, wherein the gesture performed via the instrument comprises a motion of the instrument.

9. (canceled)

10. The device of claim 1, wherein recognizing the gesture performed via the instrument comprises determining a trajectory of the instrument and a state of a procedure during which the trajectory of the instrument occurred.

11-12. (canceled)

13. The device of claim 1, wherein the gesture comprises a movement of the instrument that is distinct from movement of the instrument that occurs during execution of a procedure being performed using the instrument.

14-15. (canceled)

16. The device of claim 1, wherein the processing system, when executing the application, is further configured to prompt the operator for confirmation of the change from the first mode of operation to the second mode of operation.

17-19. (canceled)

20. The device of claim 1, wherein the change from the first mode of operation to the second mode of operation comprises changing an action performed by the instrument.

21-25. (canceled)

26. The device of claim 1, wherein the processing system, when executing the application, is further configured to:
obtain one or more of second vision data associated with the instrument, second kinematics data associated with at least one of the structure or the instrument, or second events data associated with at least one of the structure or the instrument;
determine, based on the one or more of the second vision data, the second kinematics data, or the second events data, that no gesture has been performed via the instrument; and
in response to determining that no gesture has been performed, maintain the second mode of operation.

27-28. (canceled)

29. A method comprising:
obtaining, by a processor system, kinematics data associated with at least one of an instrument or a structure of a computer-assisted device supporting the instrument, the instrument for performing tasks at a worksite in response to manipulation of a leader input device by an operator;
based on at least the kinematics data, recognizing, by the processor system, a gesture performed via the instrument; and
in response to recognizing the gesture, causing, by the processor system, a change from a first mode of operation to a second mode of operation.

30. The method of claim 29, further comprising:
obtaining, by the processor system, vision data associated with the instrument; and
recognizing, by the processor system, the gesture based further on the vision data.

31. (canceled)

32. The method of claim 29, wherein the kinematics data comprises at least one of a position, an angle, an orientation, a speed, or a velocity of at least one of the structure or the instrument.

33. (canceled)

34. The method of claim 29, further comprising:
obtaining, by the processor system, events data associated with a least one of the structure or the instrument; and
recognizing, by the processor system, the gesture further based on the events data.

35-53. (canceled)

54. The method of claim 29 further comprising:
obtaining, by the processor system, one or more of second vision data associated with the instrument, second kinematics data associated with at least one of the structure or the instrument, or second events data associated with at least one of the structure or the instrument;
determining, by the processor system based on the one or more of the second vision data, the second kinematics data, or the second events data, that no gesture has been performed via the instrument; and
in response to determining that no gesture has been performed, maintaining, by the processor system, the second mode of operation.

55-56. (canceled)

57. One or more non-transitory machine-readable media comprising a plurality of machine-readable instructions which when executed by a processor system associated with a computer-assisted system are adapted to cause the processor system to perform a method comprising:
obtaining kinematics data associated with at least one of an instrument or a structure of a computer-assisted device supporting the instrument, the instrument for performing tasks at a worksite in response to manipulation of a leader input device by an operator;
based on at least the kinematics data, recognizing a gesture performed via the instrument; and
in response to recognizing the gesture, causing a change from a first mode of operation to a second mode of operation.

58. The one or more non-transitory machine-readable media of claim 57, wherein the method further comprises:
obtaining vision data associated with the instrument; and
recognizing the gesture based further on the vision data.

59. The one or more non-transitory machine-readable media of claim 57, wherein the method further comprises:
obtaining events data associated with a least one of the structure or the instrument; and
recognizing the gesture further based on the events data.
